(19) United States
(12) Patent Application Publication
(10) Pub. No.: US 2026/0248507 A1
(43) Pub. Date: Aug. 27, 2026

(54) PERCUTANEOUS ACCESS GRAFT SYSTEM AND METHOD

(71) Applicants: UNIVERSITY OF MARYLAND, BALTIMORE, Baltimore, MD (US); UNIVERSITY OF MARYLAND MEDICAL SYSTEM CORPORATION, Linthicum, MD (US)

(72) Inventors: Aakash Yogesh SHAH, Laurel, MD (US); Bartley GRIFFITH, Gibson Island, MD (US); Zhongjun WU, Marriottsville, MD (US); Ryan SMITH, College Park, MD (US)

(21) Appl. No.: 19/162,122

(22) PCT Filed: Mar. 11, 2024
(86) PCT No.: PCT/US2024/019388

§ 371 Date: Sep. 4, 2025

Related U.S. Application Data
(60) Provisional application No. 63/451,288, filed on Mar. 10, 2023.
(60) Provisional application No. 63/536,193, filed on Sep. 1, 2023.

(51) Int. Cl. A61B 17/11; A61B 17/12

(52) U.S. Cl. CPC A61B 17/11; A61B 17/12013; A61B 2017/1107; A61B 2017/1132

(57) ABSTRACT

A method and system provide an end-to-side anastomosis graft via percutaneous access for cardiac and vascular surgeries. The system includes a vascular graft configured for stable placement within a vessel, a sheath for long-term maintenance and use of the graft, and a closure device configured for secure ligation and transection of the graft when no longer in use. The system facilitates the use of extracorporeal life support devices and other cardiac interventions, reducing the need for open surgical exposure and allowing use by non-surgeons, such as interventional cardiologists.

### CROSS-REFERENCE TO RELATED APPLICATIONS

This application claims the benefit of U.S. Provisional Patent Application No. 63/451,288 titled “PERCUTANEOUS ACCESS GRAFT,” filed by the inventors herein on Mar. 10, 2023, and of U.S. Provisional Patent Application No. 63/536,193 titled “PERCUTANEOUS ACCESS GRAFT SYSTEM,” filed by the inventors herein on Sep. 1, 2023, the specifications of which are incorporated herein by reference in their entireties.

### FIELD OF THE INVENTION

The present invention relates generally to medical devices and methods used in vascular surgery and interventional cardiology. More specifically, the invention relates to a system and method for establishing and maintaining percutaneous access to blood vessels for various medical procedures.

### BACKGROUND

The most common peripheral access sites in cardiac surgery (such as during cardiac bypass procedures and transcatheter interventions) are the axillary artery and the femoral artery. When comparing the two sites, the femoral artery is on average larger with a median size of 8 mm compared to 6 mm in the axillary artery, and the distribution of axillary artery diameter are generally smaller than 8 mm. The axillary artery is more challenging of the two access sites.

Cannulation for axillary artery for bypass typically includes directly cannulating or sewing on a side graft. Directly cannulating can be faster, but can also carry a higher incidence of complications, such as dissection from manipulation of a large bore device in a small artery, stroke (presumably from occlusion of vertebral artery branches), and distal extremity ischemia. In both approaches a cutdown is performed to get control of the artery and for ease of removal after use. Direct cannulation to deal with the distal extremity typically includes the use of a distal perfusion catheter, just like in the case of the femoral artery, particularly for long-term access typical of ECMO procedures.

Although the side graft technique is typically more reliable, it has many shortcomings. These include requiring surgical access to sew the graft, which even in skilled hands can take 20-30 minutes, and repeated access to transect and ligate the graft after use. The use of grafts in an end-to-side fashion requires an incision and surgical exposure of the vessel, and direct suturing of the graft to the vessel. This carries the risk of infection of the incision site, inadvertent damage to surrounding structures during exposure of the vessel, time required to expose the vessel and suture the graft, and surgical expertise.

Current alternatives, such as standard grafts, require open surgical exposure of the vessel. Further, large bore sheaths may be used as a site of access; however, they may inhibit distal flow within vessels leading to thrombosis or ischemia of the tissue supplied by the distal vascular bed. Placement of an end-to-side graft currently requires an incision and surgical exposure of the desired vessel, isolation, clamping, and opening of the vessel to allow the graft to be directly sutured to the vessel. If the graft is intended for use in the setting of extracorporeal life support, then a tubing connector or cannula is placed into the graft and secured in place by tying sutures around the graft. If the graft is intended for use as an access site for other device insertion, the graft is trimmed to remain at the level below the skin, and a sheath is placed within the graft and secured in place by tying sutures around the graft. Once the graft is no longer being used, the graft is exposed again through the incision and ligated near the site of anastomosis with the vessel with either surgical slips, suture, or staples and transected.

Thus, there remains a need in the art for timely and better life-saving interventions for patients suffering from heart failure and severe complications of cardiovascular disease, (CVD), and in particular for a system that reduces the complexity and clinical skill required for end-to-side anastomosis, mitigates the complications associated with open surgical exposure, and allows for the procedure to be performed by non-surgeons.

### SUMMARY OF THE INVENTION

Provided herein according to several exemplary configurations is a novel system and method for reducing the complexity and clinical skill required for an end-to-side anastomosis with a percutaneous access graft that avoids one or more disadvantages of prior art systems and methods. The system and method are configured to make such procedures easier for surgeons and mitigate the complications these patients often experience with open surgical exposure of vessels for graft anastomosis, compared to typical devices. In addition, the system and method are configured to allow non-surgeons (e.g., interventional cardiologists who manage these patients) to perform these life-saving procedures. Specifically, a percutaneous access graft configured in accordance with aspects of the invention is intended for placement in vessels via a Seldinger technique in a configuration like an end-to-side surgical anastomosis that can be maintained for an extended period and support various life-saving procedures. This can be used as a conduit for institution of extracorporeal life support devices (e.g., cardiopulmonary bypass, extracorporeal membrane oxygenation), or as an insertion site for other devices (e.g., intra-aortic balloon pump, transcatheter aortic valve insertion, percutaneous ventricular assist device insertion). These procedures are performed with increasing frequency. These procedures are performed as an open surgery that is time-intensive and fraught with a high complication rate.

According to several exemplary configurations, and further described herein, the system includes an access graft, a sheath, and a closure device. Systems configured in accordance with aspects of the invention may include many features configured to alleviate the technical issues noted above with previously known systems and methods, and may improve care for patients. In exemplary configurations, the system includes a vascular graft configured to be percutaneously placed. The graft can include a portion having a flared lip for being placed in a vessel and a stented portion (or segment) for positioning proximal to the vessel that is being accessed. The stented portion of the graft is configured to withstand radial forces at the entrance site of the vessel and/or in the overlying soft tissue to keep the graft in place and provide hemostasis. The access graft is configured to be inserted into a vessel percutaneously or via surgical exposure.

Still further, a graft according to aspects of an embodiment is illustrated in FIG. 29 being inserted into an artery model, and shows a flared end configured to maintain the position of the access graft. In certain configurations, the flared end maintains the position of the graft by providing a frictional force on a blood vessel. Still further, the flared end can provide a frictional force on multiple portions of a blood vessel. For example, a first flared portion on a bottom side is configured to sit inside of a blood vessel and a second flared portion is configured to be positioned above the first flared portion to provide a frictional force from the outside of the blood vessel, thus “sandwiching” the blood vessel between the two flared portions. The flared end provides stability to the graft and may provide an additional measure for hemostasis. Thus, the skill-level required to attach a graft to a vessel, permitting use of the system for life-saving cardiac procedures by both surgeons and non-surgeons outside of the operating room (OR) is reduced, compared to typical devices and methods.

Still further, the system according to further aspects of an embodiment can include a sheath designed for long-term maintenance of the percutaneous access graft. The sheath includes an outer portion (e.g., placed outside the access graft) and an inner portion (placed within the access graft) to maintain access of the access graft. The two portions of the sheath are configured to be removably coupled to one another to prevent exposure of the access graft to the environment, thereby reducing the risk of infection during long-term use, compared to typical devices.

Still yet further, the system according to further aspects of an embodiment can include a closure device. The closure devices is configured to close the access graft, such as when the access graft is no longer in use. According to an exemplary configuration, the closure device includes a shaft configured to pass the access graft, such as into a patient or near a vessel. The closure device is further configured to close (or ligate) the access graft, such as by compressing a collet (e.g., metal collar). Furthermore, in an exemplary configuration the closure device may be configured to remove a portion of the access graft, such as an excess portion of the access graft that is above the collet. For example, once hemostasis is confirmed the closure device can cut (or transect) the access graft above the collar. Currently, such operations require a surgeon to re-open an incision in and use a vascular stapler on a typical graft or clamp it above the artery and stitch it shut.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1A is a cross-section view of a percutaneous access graft closure device, according to several embodiments;

FIG. 1B is a side view of the percutaneous access graft closure device of FIG. 1A;

FIG. 1C is a cross-section view of a percutaneous access graft closure device, according to another embodiment, including a collet;

FIG. 1D is a diagram of an exploded view of the closure device of FIG. 1C;

FIG. 2 is a diagram that illustrates the device of FIG. 1A with respect to a percutaneous graft;

FIG. 3 is a drawing of a percutaneous access graft, having been ligated, inserted using the device of FIG. 1A in partial cross-section;

FIG. 4A is an illustration of a cross-sectional view of the device of FIG. 1A showing closure jaws in an open position;

FIG. 4B is an illustration of a cross-sectional view of the device of FIG. 1A showing closure jaws in a closed position;

FIG. 5A is an illustration of a perspective view of the device of FIG. 1A showing closure jaws in an open position;

FIG. 5B is an illustration of a perspective view of the device of FIG. 1A showing closure jaws in a closed position;

FIG. 5C is a set of drawings illustrating the closure jaws of FIG. 1A in an open position (left) and a closed position (right);

FIG. 6 is a drawing of a side view of a collet, according to several embodiments, such as shown in FIG. 1C;

FIG. 7A is a drawing of a top view of the collet shown in FIG. 6;

FIG. 7B is a set of drawings illustrating multiple views of the collet shown in FIG. 6, including the collet in an open state and a crushed state;

FIG. 8 is an illustration of a front view of one side of a closure jaw, according to several embodiments;

FIG. 9 is an illustration of a side view of one side of the closure jaw shown in FIG. 8;

FIG. 10 is a close-up front view of one side of a closure jaw, shown in FIG. 8 showing gripping teeth in a diamond pattern at a distal end of the closure jaw, according to several embodiments;

FIG. 11 is a drawing illustrating percutaneously accessing a blood vessel and placing a guidewire, according to one embodiment of a method of using the device of FIG. 1A;

FIG. 12 is a drawing illustrating inserting a sheath, according to one embodiment of a method of using the device of FIG. 1A;

FIG. 13. is a drawing illustrating inserting a percutaneous access graft through the sheath of FIG. 12 and deploying a flared portion of a graft within a blood vessel;

FIG. 14A is a drawing illustrating a deployed percutaneous access graft in a blood vessel;

FIG. 14B is an image of a deployed percutaneous access graft, such as shown in FIG. 14A:

FIG. 15A is a drawing of an alternative embodiment of a percutaneous access graft being configured as a stand-alone graft

FIG. 15B is a drawing of another alternative embodiment of a percutaneous access graft being configured to attach to a tubing connector or a cannula for bypass or ECMO purposes;

FIG. 15C is a drawing of yet another alternative embodiment of a percutaneous access graft having a specially-designed sheath for long-term access;

FIG. 16 is an image of an alternative percutaneous access graft, such as shown in FIG. 15B, attached to a tubing connector;

FIG. 17 is an image of an alternative percutaneous access graft, such as shown in FIG. 15B, attached to a tubing connector and external cannula;

FIG. 18 is a drawing of a deployed percutaneous access graft connecting to tubing, such as shown in FIG. 15B, and a close-up cross-section of a tubing connector cap;

FIG. 19A is an image of a percutaneous access graft having a sheath, such as shown in FIG. 15C, in which the sheath is advanced along the graft;

FIG. 19B is an image of a percutaneous access graft having a sheath, such as shown in FIG. 15C, in which the sheath is retracted toward a proximal end of the graft (e.g., partially outside of a patient's skin)

FIG. 19C is an image of a percutaneous access graft having a sheath, such as shown in FIG. 15C, and an inner sheath;

FIG. 19D is an image of a percutaneous access graft having a sheath, such as shown in FIG. 19C, and an inner sheath in which the inner sheath is coupled to the outer sheath through the graft;

FIG. 20A is a drawing of a percutaneous access graft positioned in a collet, such as shown in FIGS. 6 and 7, in which the collet is in an open state;

FIG. 20B is a drawing of a percutaneous access graft positioned in a collet in which the collet is in a ligated state;

FIG. 20C is a drawing of a transected percutaneous access graft positioned in a collet in which the collet is in a ligated state;

FIG. 21 is an image showing a side view of a ligated percutaneous access graft positioned in a collet in which the collet is in a ligated state;

FIG. 22 is an image showing a side view of a ligated percutaneous access graft positioned in a collet in which the collet is in a ligated state;

FIG. 23A is an image of a percutaneous access graft having a sheath, such as shown in FIG. 19C, and an inner sheath;

FIG. 23B is an image of a percutaneous access graft having a sheath and an inner sheath, such as shown in FIG. 23A, in which the inner sheath is coupled to the outer sheath through the graft;

FIG. 24A is a schematic drawing of a cross-sectional view of a closure device of one embodiment of a percutaneous access graft device; and

FIG. 24B is a schematic drawing of a cross-sectional view of a closure device of another embodiment of a percutaneous access graft device.

FIG. 25A is an image of one embodiment of the closure device of FIG. 1A in an open state;

FIG. 25B is an image of one embodiment of the closure device of FIG. 1A in a closed state;

FIG. 26 is a set of drawings of one embodiment of the device of FIG. 1A;

FIG. 27 is a set of drawings of one embodiment of the device of FIG. 1A;

FIG. 28 is a set of drawings of a spacer of the device of IA;

FIG. 29 is a set of images depicting one example of the percutaneous access graft, according to one embodiment, having a dual flared portion being deployed;

FIG. 30 is a set of images depicting one example of the sheath being deployed; and

FIG. 31 is a set of images depicting one example of the closure device transecting a percutaneous access graft.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

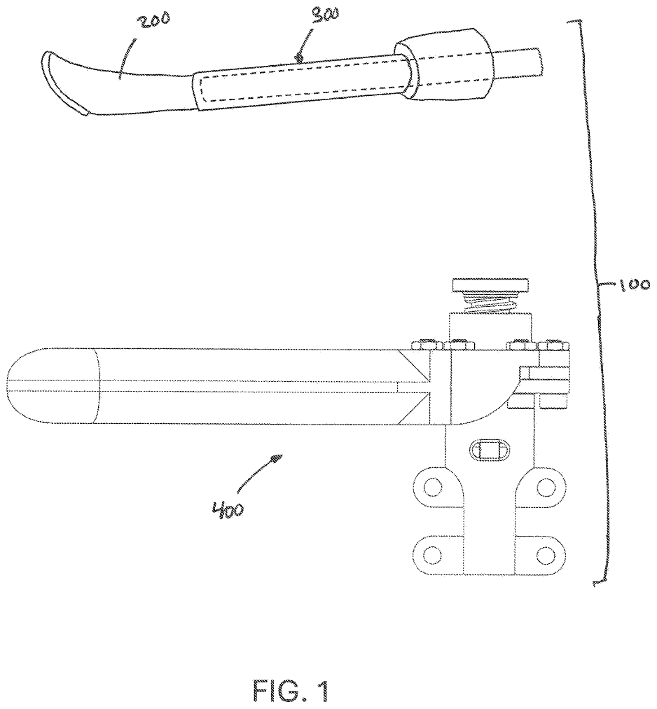

In accordance with certain aspects of an embodiment, and with particular reference to the FIG. 1, a system 100 for percutaneous insertion, maintenance and ligation of an access graft 200 is provided. System 100 is configured to simplify the process of vascular access in cardiac and vascular surgeries, and may be particularly beneficial for patients suffering from heart failure or severe complications of cardiovascular disease, where timely and less invasive interventions are critical.

To meet these goals, system 100 according to certain aspects of an embodiment includes graft 200, sheath 300 (preferably including an outer sheath 330 that is configured to surround an outer portion of graft 200, and inner sheath 350 configured to extend into graft 200 and that may be locked to outer sheath 300 so that graft 200 is captured between outer sheath 300 and inner sheath 350), and closure device 400 that may be used to ligate graft 200 once the procedure requiring graft 200 has been completed. Potential users of system 100 may include centers that perform cardiac surgery, vascular surgery, and interventional cardiology with placement of percutaneous assist devices, such as intra-aortic balloon pumps and percutaneous ventricular assist devices. In such applications, system 100 may be used, for example, to assist with placement of an axillary Impella, with cardiac bypass procedures, and with ECMO applications. Likewise, system 100 may be further configured for transcatheter interventions, such as TAVR or TEVAR, in which femoral access is not feasible, or temporary support such as a balloon pump, in which axillary use is more common with a revised allocation system.

System 100 is thus configured for use in the setting of extracorporeal life support devices (e.g., cardiopulmonary bypass, or extracorporeal membrane oxygenation) or for the temporary establishment of an access site for the intended vessel in the setting of catheter interventions or the insertion of other devices (e.g., percutaneous ventricular assist devices) where a large caliber in-dwelling sheath is not feasible. Exemplary embodiments of the invention may comprise system 100 including access graft 200, sheath 300, and closure device 400. Other exemplary embodiments may comprise access graft 200 with or without sheath 300. Still other exemplary embodiments of the invention may comprise closure device 400.

The access graft system 100 may allow attachment of access graft 200 to a vessel with less surgical exposure than typical methods require, may shorten the time required for providing arterial access, and may allow those with limited surgical expertise, and preferably even those without surgical expertise, to more easily place access graft 200 in comparison to typical procedures. Further, ligation and transection of grafts currently requires surgical exposure of the graft, after which the graft can be closed or sealed, such as clipped, stapled, or oversewn. The access graft system 100 is further configured to enable ligation and transection of the access graft with less surgical exposure than typical methods, to shorten the time required for such procedures, and to again allow those with limited or without any surgical expertise to be able to carry out such processes more easily in comparison to typical graft ligation and transection procedures.

In exemplary configurations of system 100, percutaneous access graft 200 is configured for placement in vessels via a Seldinger technique to be maintained in a configuration like an end-to-side surgical anastomosis. This configuration may be used as a conduit for institution of extracorporeal life support devices (e.g., cardiopulmonary bypass, extracorporeal membrane oxygenation), or as a site of access for insertion of other devices (e.g., transcatheter aortic valve insertion, percutaneous ventricular assist device insertion). Several embodiments of the invention may be designed to suit specific needs.

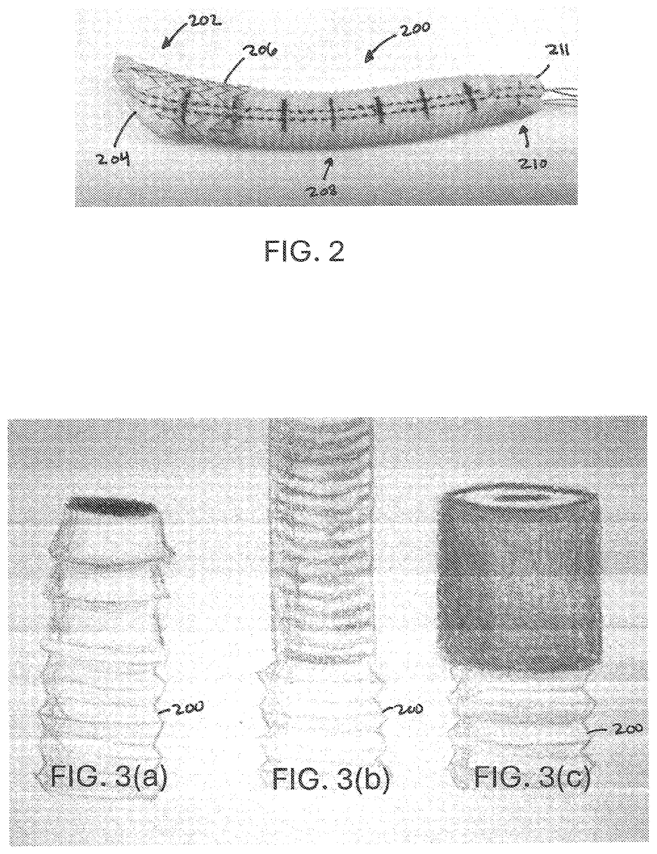

With particular reference to FIG. 2, access graft 200 comprises a vascular graft having a tubular structure configured for percutaneous placement into a vessel using the Seldinger technique. Access graft 200 includes a distal portion 202 that enters the vessel and that includes a flared lip 204 that, upon deployment, expands to engage the interior and exterior surfaces of the vessel wall. This dual engagement provides stability and hemostasis, reducing the need for extensive surgical exposure. Access graft 200 also includes a stented portion 206 adjacent to flared lip 204 that is configured to be located proximal to the vessel after placement. In exemplary configurations, the flared lip 204 may be oriented at a 90-degree angle to the longitudinal axis of graft 200. In other exemplary configurations, varying degrees of orientation of flared lip 204 with respect to graft 200 may be provided to accommodate for the angle of approach and intended purpose for which graft 200 is to be used (e.g., a 45-degree angle to promote blood flow preferentially in one direction to the attached vessel, or to allow wires and devices to insert through the graft into the vessel at a less acute angle). The stented portion 206 of access graft 200 may be configured to withstand radial forces at the entrance site of the vessel and/or in the soft tissue superficial to the vessel to keep graft 200 in place and provide hemostasis. Stented portion 206 is preferably configured to accommodate various vessel sizes and shapes, allowing for a secure fit and minimizing the risk of migration or leakage.

In certain exemplary configurations, central portion 208 and proximal portion 210 of access graft 200 are unstented. This allows for ease of use for a percutaneous closure device 400 (such as described below).

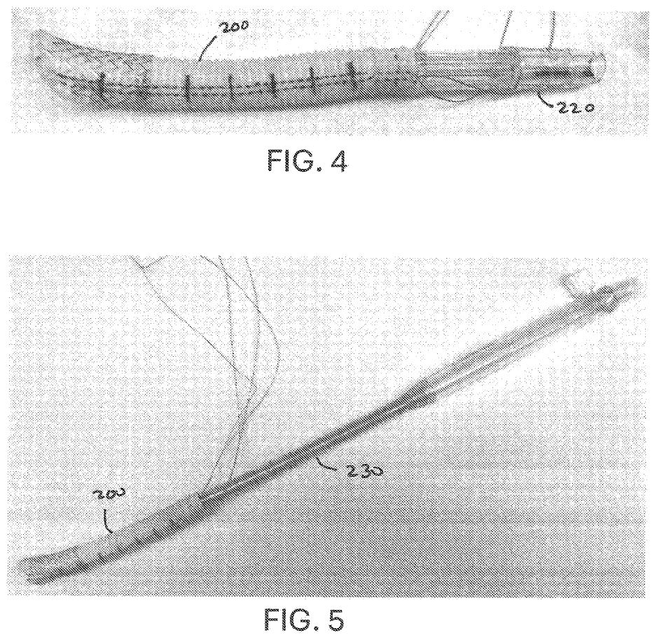

With particular reference to FIGS. 3(a) through 3(c), the proximal end 211 of graft 200 (i.e., the end proximal portion 210 of graft 200 that is opposite to the end that enters the vessel) may be configured for use with a variety of procedures. By way of non-limiting example and as shown in FIG. 3(a), in exemplary configurations access grant 200 may comprise a standalone vascular graft configured for use with a typical tubing connector inserted for connection to extracorporeal life support, a cannula, or other devices that may be inserted and tied to the graft. FIG. 4 provides a further detailed view of graft 200 affixed to a tubing connector 220. In another exemplary configuration and as shown in FIG. 3(b), access graft 200 may include a typical tubing connecter or arterial cannula coupled to the proximal end for ease of connection to cardiac bypass or extracorporeal life support devices. FIG. 5 provide a further detailed view of graft 200 affixed to a tubing connector with an external cannula 230. In yet another exemplary configuration, access graft 200 may be provided with sheath 300 (as described in greater detail below) when intended for use in long-term maintenance. In certain exemplary configurations, proximal end 111 can include a second flare on proximal end 211 with suture loops, or a metal ring to lock into sheath 300.

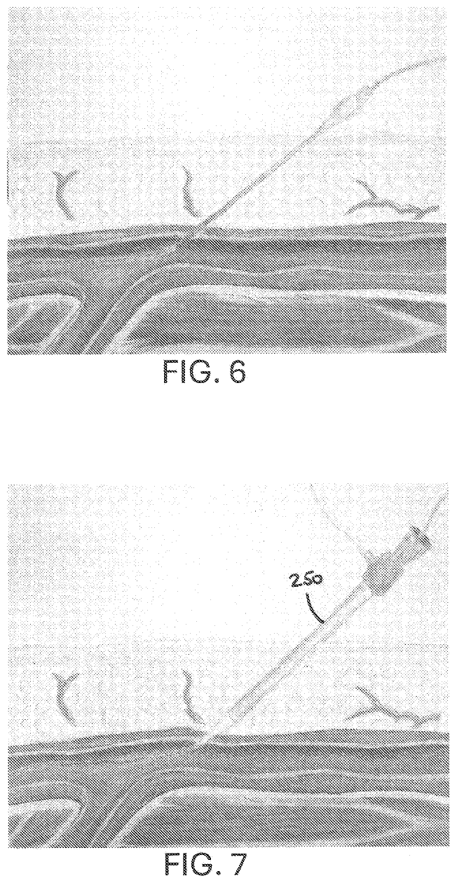

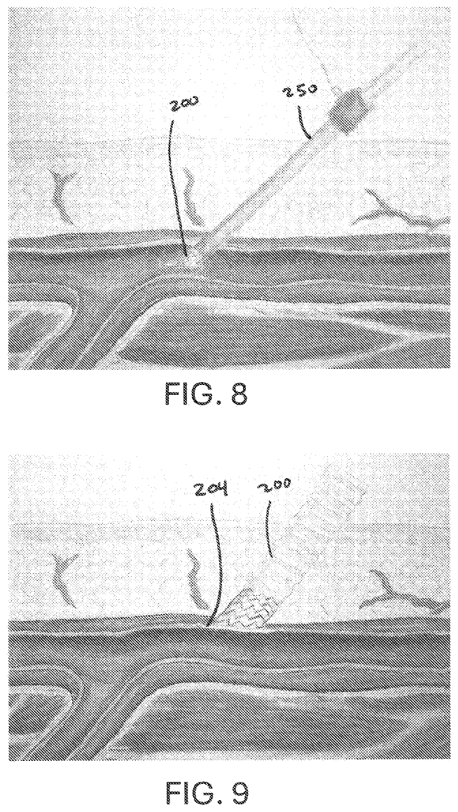

As noted above, graft 200 is configured for percutaneous insertion into a vessel. In an exemplary configuration, graft 200 is inserted into a vessel by first percutaneously accessing the vessel (e.g., an artery) at an arteriotomy site and introducing a guidewire 240 into the vessel, as shown particularly in FIG. 6, after which a standard sheath 250 having a diameter that is smaller than the fully expanded stent portion of the graft is percutaneously inserted into the vessel, as shown in FIG. 7. Graft 200 may then be introduced into a vessel through the standard sheath 250, as shown in FIG. 8, and the flared lip 204 deployed from standard sheath 250 into an open state in the vessel, the flared lip 204 then being retracted such that flared lip 204 couples to the vessel, such as by abutting the vessel wall at the arteriotomy site. An outer portion of the graft 200 can include markings to more efficiently determine a depth at which the graft is attached to the vessel. As shown in FIG. 9, standard sheath 250 may then be removed, leaving graft 220 fully deployed in the vessel and providing access to the vessel. In this manner, system 100 may be configured to allow improved flow to a distal extremity by reducing obstruction to potentially important branches that might result from use of a larger sheath or cannula left in place, or from use of a larger access device relative to the size of a vessel, such as by using a completely intravascular sheath.

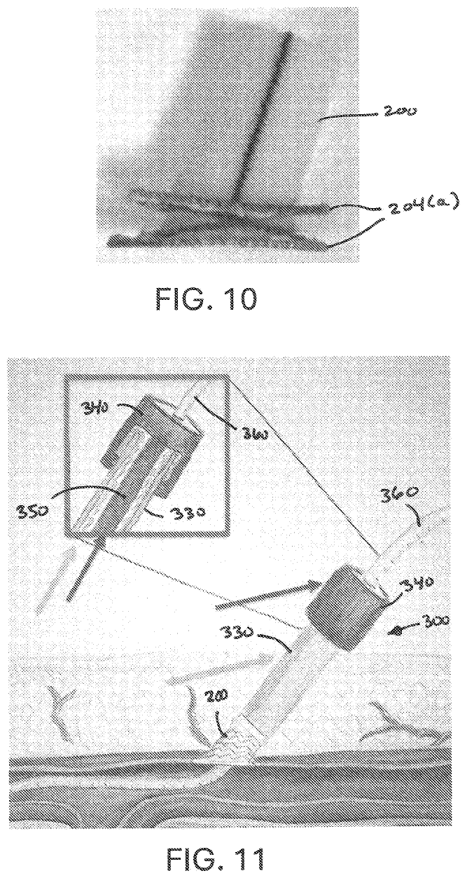

In certain configurations and as shown in FIG. 10, graft 200 may include a double-walled flared lip 204(a) having a distal portion configured to sit inside of the vessel against the interior of the vessel wall, and a proximal portion configured to sit outside of the vessel against the exterior of the vessel wall. In this configuration, the flared end of graft 200 provides a frictional force on multiple portions of the blood vessel, effectively “sandwiching” the vessel wall between the two flared sections of double-walled flared lip 204(a). This configuration may enhance the stability of the graft and provide an additional measure of hemostasis.

In accordance with certain aspects of an embodiment of the invention, and with reference again to FIG. 1 and to FIG. 11 showing graft 200 deployed with sheath 300, sheath 300 may be provided for use with graft 200, particularly for long-term use and maintenance of graft 200 for providing access to a vessel. In an exemplary configuration, sheath 300 is configured for use as an access site for various interventions or device insertions and to maintain sterility for the portion of access graft 200 that is above the skin. Sheath 300 preferably provides a dual-layer assembly comprising outer sheath 330 and inner sheath 350. Outer sheath 330 of sheath 300 is configured to receive the distal portion of graft 200. For example, a portion of outer sheath 330 is configured to be inserted below the skin, and a portion of outer sheath 330 is likewise configured to remain above the skin. Thus, the outer sheath is configured to reduce the likelihood of exposure of graft 200 to the environment during long-term use. Inner sheath 350 is configured to be placed within graft 200 and coupled to the outer sheath 330, such as by way of a locking cap 340. Inner sheath 350 is configured to further allow access or maintenance of other devices, such as by way of non-limiting example additional tubing 360, to be inserted through graft 200 into the vessel. Graft 200 is configured to be secured in a desired position, such as described above. For example, graft 200 can be secured between outer sheath 330 and inner sheath 350 to reduce the likelihood of graft 200 moving from a desired position and to maintain hemostasis. In an exemplary configuration, locking cap 340 may lock outer sheath and inner sheath together to maintain the access graft in a desired position and maintain hemostasis.

FIG. 12 is a schematic view of the sequence for percutaneous deployment of sheath 300. In the deployment model shown in FIG. 12, percutaneous access graft 200 has been placed, and an external device (e.g., Impella) has been inserted through access graft 200 into the vessel. In pane (A), outer sheath 330 is loaded above access graft 200, and outer sheath 330 is advanced down around the outside of the graft. Preferably, sutures at a proximal end of graft 200 may be grasped and initially fed proximally through the outer sheath 330 and pulled taught, thus keeping graft 200 taught as outer sheath 300 is advanced over graft 200. In pane (B), inner sheath 350 is then advanced down through the interior of access graft 200. In pane (C), inner sheath 350 and outer sheath 330 are then locked together using locking cap 340.

In accordance with still further aspects of an embodiment, system 100 may include closure device 400 (FIG. 1) to ligate graft 200 after the need for access to the vessel has ended and all external devices have been removed. Closure device 400 includes a body section 410 defining a shaft 412 configured to allow graft 200 to pass through the interior of the shaft 412 from a point near to the accessed vessel. Preferably, sutures that remain at the proximal end of graft 200 may be fed through shaft 412 of closure device 400 (as discussed in greater detail below) and pulled to keep graft 200 taught as ligation proceeds. Outer body portion 410 of closure device 400 may optionally include markings to indicate an insertion depth of closure device 400, such that closure device 400 can be operated at a desired location along access graft 200 (e.g., near an attachment location to a vessel).

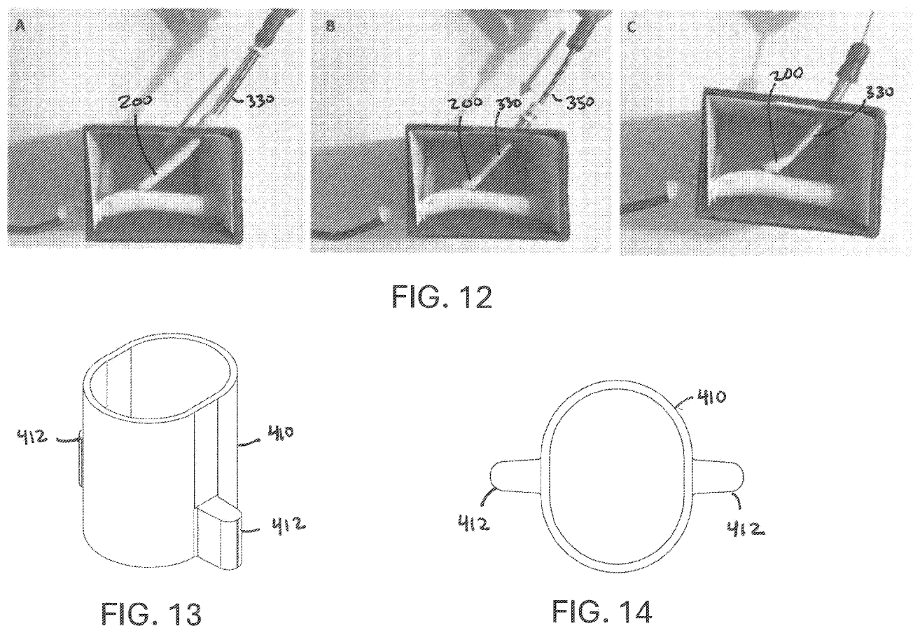

In an exemplary configuration, closure device 400 is configured to close access graft 200 with a variety of fasteners, such as a collet 410 (such as, for example, a metal collet) as shown in FIG. 13. The collet 410 may be comprised of many materials, such as metal, plastic, or ceramic. Collet 410 may likewise be of a variety of shapes (e.g., circular, oval, elliptical), and can have a variety of inner surfaces (e.g., smooth metal, felt lining, polytetrafluoroethylene lining, metal protrusions) to increase the ease, secureness, and closure of the access graft. For example, the inner surface of collet 410 may increase the secureness of the access graft 200 by increasing friction, such as by having knurling or other like. Closure device 400 is configured to compress the collet 410 by closing or compressing graft 200 to maintain hemostasis. The outer shaft of closure device 400 generally has cross-sectional dimensions that are smaller than the diameter of graft 200 and outer sheath 330. This allows for closure device 400 to be used through the same tract in tissue in which access graft 200 is placed, or with very limited exposure of graft 200 beneath the skin.

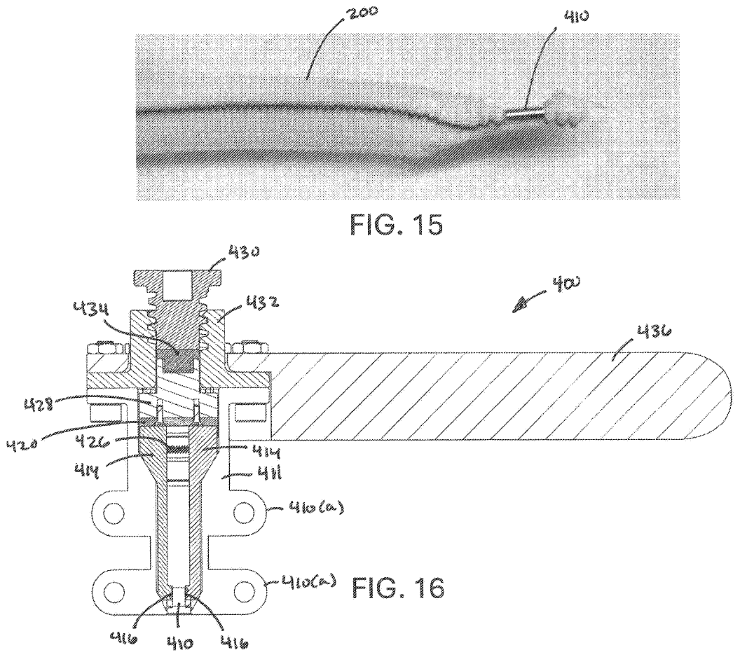

As shown in FIGS. 13 and 14, collet 410 may, in an exemplary configuration, be formed as a generally oblong cylinder having outwardly extending arms 412 extending outward from the bottom exterior of collet 410. Arms 412 are configured to engage (as discussed in greater detail below) crimp jaws of closure device 410 to collapse collet 410 onto access graft 200 at the location at which it is to be ligated. In this manner, the exterior walls of collet 410 adjacent to arms 412 are pushed towards one another with an end of access graft 200 extending through the hollow interior of collet 410, crushing the collet 410 onto access graft 200 to maintain hemostasis, as shown in FIG. 15.

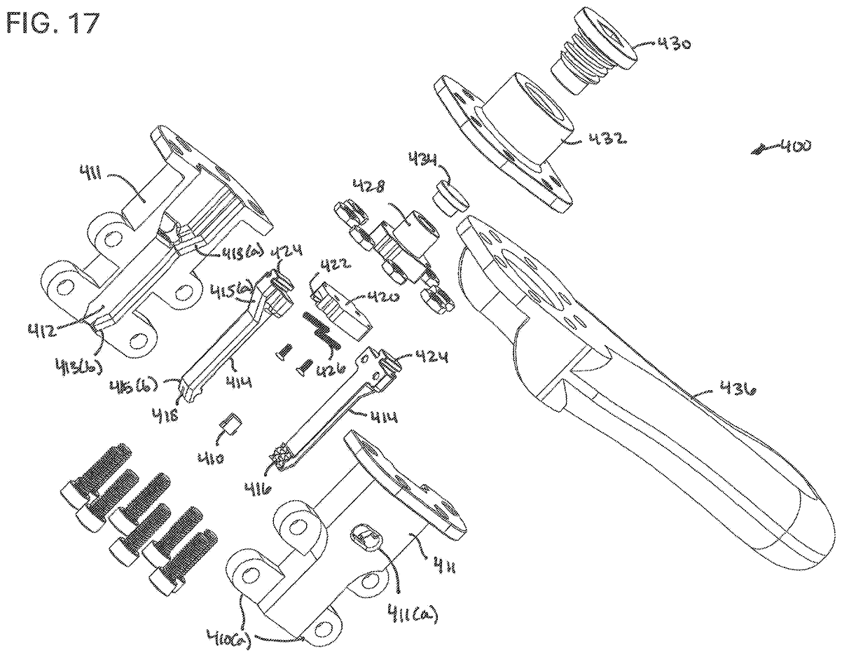

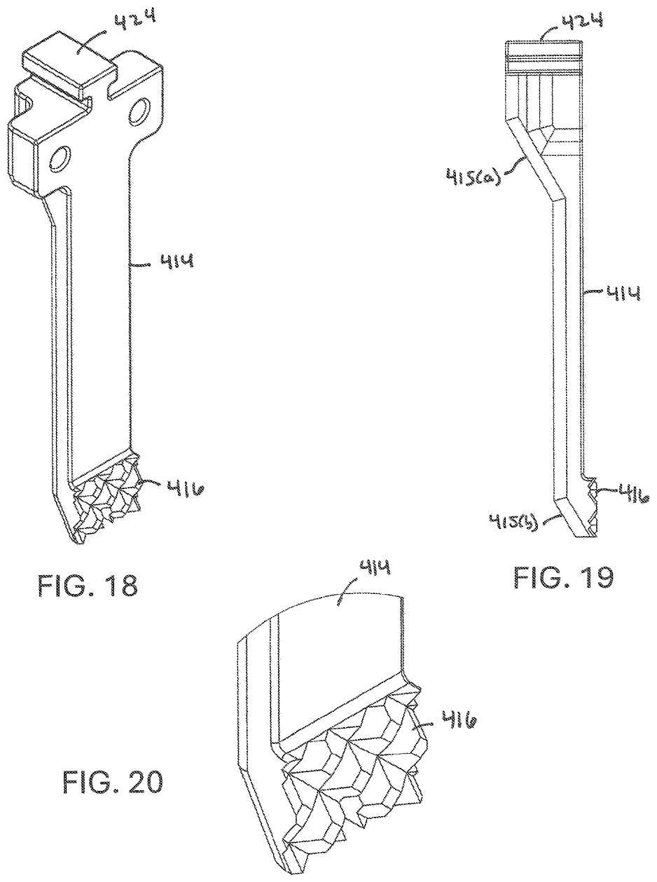

Closure device 400 is configured to allow an operator to close access graft 200 in narrow and sterile environments, such as percutaneous openings of a patient. Closure device 400, as further described below, provides sufficient force to close access graft 200 by crushing collet 410 percutaneously in a patient using minimal force (e.g., manual, hand force) and minimizing risk to adjacent tissue. In order to enable closure device 400 to close access graft 200 with collet 410, and with particular reference to FIGS. 16-20, two crimp jaws 414 are provided inside of shaft 412 that feature an interlocking set of teeth 416 on the bottom, distal end of each crimp jaw 414. Crimp jaws 414 sit inside of shaft 412 of body portion 410 with angled features designed to support the backs of the jaw while crushing the collar. More particularly, shaft 412 has narrowing sections 413(a) and 413(b), and crimp jaws 414 have mating angled outer surfaces 415(a) and 415(b) that engage with narrowing sections 413(a) and 413(b) of channel 412, respectively. Thus, as crimp jaws 414 are pushed downward in channel 412, narrowing sections 413(a) and 413(b) push angled outer surfaces 415(a) and 415(b) inward, in turn bringing crimp jaws 414 toward one another.

Each crimp jaw 414 has a collet receiver slot 418, each of which is configured to receive an outwardly extending arm 412 of collet 410 to hold collet 410 in place between crimp jaws 414 as it is being crushed around access graft 200.

Closure device 400 includes a drive mechanism for causing clamp jaws 414 to crush collet 410 around access graft 200. The drive mechanism preferably includes a channel plate 420 that directly receives the top of each closure jaw 414. Channel plate 420 includes a channel 422 on its underside that receives a mating slide 424 on the top of each crimp jaw 414, thus allowing each crimp jaw 414 to slide toward and away from one another during a closure operation. Preferably, one or more return springs 426 are provided between interior faces of crimp jaws 414 biasing each crimp jaw 414 outward (i.e., away from one another). A plunger 428 is positioned above channel plate 420 and is slidable in body portion 410 along the longitudinal axis of shaft 412. Thus, as plunger 428 is pushed downward, it pushes channel plate 420 downward, causing narrowing sections 413(a) and 413(b) of shaft 412 to push against angled outer surfaces 415(a) and 415(b) of crimp jaws 414 and move them toward one another against the bias of springs 426 to crush collet 410 around an access graft 200 that extends through collet 410 into shaft 412. Of course, while channel plate 420 and plunger 428 are shown as separate components, they may comprise a single, unitary assembly without departing from the scope of the invention.

Preferably, a threaded piston 430 may be provided for access from the top of closure device 400, threaded piston 430 being threaded into a cover plate 432 that is positioned over plunger 428. Thus, when a closure operation is to be performed, an operator may rotate threaded piston 430 using a mating driver, moving the base of threaded piston 430 downward to engage plunger 428 and, in turn, push crimping jaws 414 toward one another. Optionally, a spacer 434 may be provided between the bottom of threaded piston 430 and plunger 428 to ensure smooth transfer of movement of threaded piston 430 to plunger 428, though spacer 434 as shown may be integrally formed with plunger 428 without departing from the scope of the invention.

A handle 436 is also provided and affixed to body portion 410 and cover plate 432 to enable manipulation of closure device 400 by an operator. Optionally, body portion 410 may be provided in separate halves for ease of manufacture, with those separate halves being joined together via threaded members or other similarly configured fasteners extending through fastening flanges 410(a) extending outward from the sides of each body portion 410.

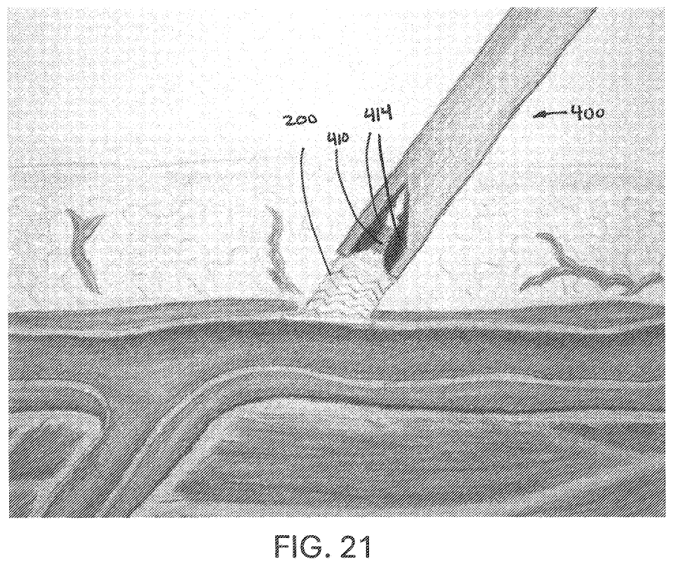

In use, and to percutaneously ligate access graft 200 that has been placed percutaneously or by surgical exposure, an uncrushed collet 410 is loaded into crimp jaws 414 with each outwardly extending arm 412 of collet 410 positioned in a collet receiver slot 418. Sutures at the proximal end of access graft 200 are grasped and the ends of those sutures are fed through collet 410, into shaft 412, and out of window 411 in body portion 410 of closure device 400. The sutures from access graft 200 may then be pulled as the body portion 410 is advanced towards the stented end of access graft 200 (i.e., the end engaging the vessel), causing the proximal end of access graft 200 to pass through the interior of collet 410. When the distal end of closure device 400 is positioned at the desired location with respect to access graft 200, an operator may then engage threaded piston 430 to compress crimp jaws 414 and crush collet 410 around access graft 200 to maintain hemostasis. Threaded piston 430 may then be rotated in the opposite direction such that crimp jaws 414 return (under the bias of springs 426) to their open position to allow crimp jaws 414 to release after crimping the collet 410. FIG. 21 shows a schematic view of a bottom portion of closure device 400 percutaneously ligating access graft 200 with collet 410.

Optionally, a percutaneous cutter 500 may be provided for transecting access graft 200 after it has been ligated. For example, percutaneous cutter may include a transection portion, such as a blade or thermal cautery, at the distal end of a shaft that may engage access graft 200 and transect access graft 200 to maintain hemostasis.

In an exemplary configuration, an in accordance with certain aspects of an embodiment, percutaneous cutter 500 may comprise an outer cylinder 502 having a cutting blade 504, an inner cylinder 512 having a cutting blade 514, and a handle 520 fixedly mounted to outer cylinder 502. Outer cylinder 502 is rotatable with respect to inner cylinder 512, such that cutting blade 504 is rotatable with respect to cutting blade 514, as discussed in greater detail below. Preferably, a one-way clutch 516 roller bearing is positioned around the exterior of inner cylinder 512, and particularly between the exterior of inner cylinder 512 and the interior of outer cylinder 502, to enable rotation of outer cylinder 502 with respect to inner cylinder 512 in a single direction. Outer cylinder 502 may be rotated in a single direction with respect to inner cylinder 512 through rotation of handle 520.

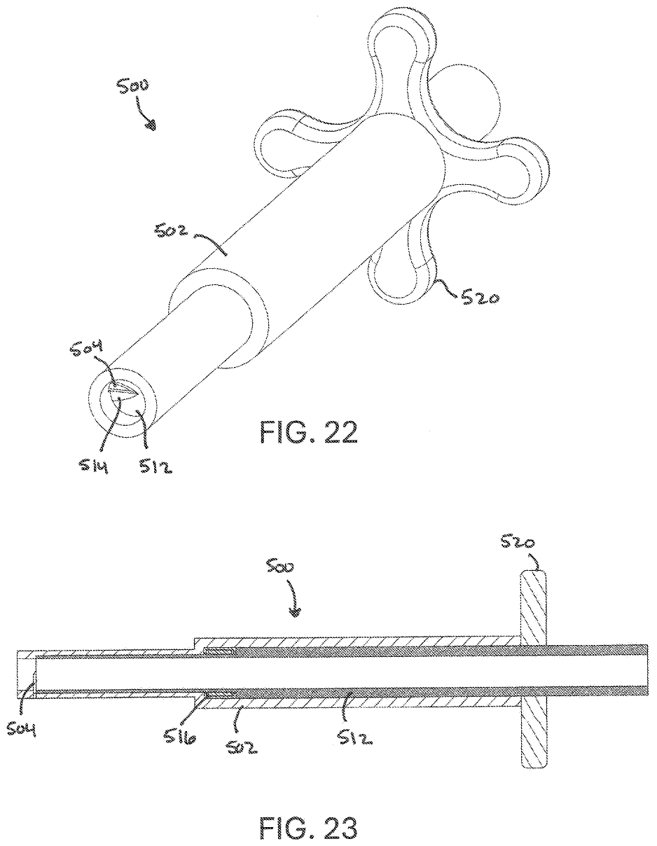

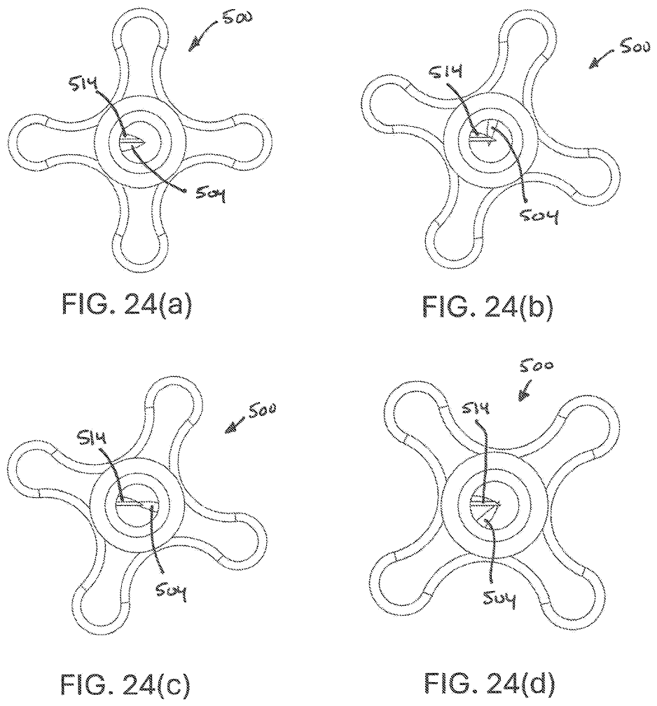

As best viewed in FIG. 22 and the variable positions of cutting blades 504 and 514, the sharpened portion of each of cutting blades 504 and 514 are positioned to face one another (best shown in FIGS. 22 and 24(a)), such that the outer edges of the cutters will not snag access graft 200 as it is placed into cutter 500.

In use, sutures extending from the proximal end of ligated access graft 200 are fed through the interior of cutter 500, past cutting blades 504 and 514, and ultimately exiting cutter 500. An operator may then pull the sutures to cause the proximal end of ligated access graft 200 into cutter 500, with the proximal end of access graft 200 positioned in the open interior portion of inner cylinder 512 (particularly the open interior space 518 inside of inner cylinder 512 adjacent to cutting blades 504 and 514). With ligated access graft 200 pulled taught via the sutures extending from the proximal end of the access graft 200, handle 520 may then be rotated to rotate outer cylinder 502, and thus cutting blade 504, with respect to cutting blade 514. Each of cutting blade 504 and cutting blade 514 has a length that is greater than the radius of the interior of inner cylinder 512 to ensure that a full 360° rotation of cutting blade 504 will engage and cut the proximal portion of access graft 200. Thus, upon rotation of handle 520, cutting blade 504 will rotate about the interior of cutter 500 through the sequence shown generally in FIGS. 24(a) through 24(d), whereby (i) as shown in FIG. 24(a), the cutting blades 504 and 514 first have their respective cutting surfaces facing one another with the access graft 200 (not shown) positioned in open interior space 518, to (ii) cutting blade 504 affixed to outer cylinder 502 sweeping 360° around open interior space 518 as outer cylinder 502 is rotated, such that the cutting surface of rotating cutting blade 504 engages access graft 200 and ultimate shears it in scissors fashion when cutting blade 504 returns to and proceeds past fixed cutting blade 514, in turn severing the proximal portion of access graft 200 above collet 410 after confirming hemostasis.

In other exemplary configurations, cutter 500 may include other mechanical blades, an electrocautery device, a laser cutter, or similarly configured cutter assemblies.

Thus, provided according to several embodiments is a percutaneous access graft system configured to place a percutaneous access graft in vessels via a Seldinger technique and to be maintained in a configuration like an end-to-side surgical anastomosis. The system can be used as a conduit for institution of extracorporeal life support devices (e.g., cardiopulmonary bypass, extracorporeal membrane oxygenation), or as a site of access for insertion of other live-saving devices (e.g., transcatheter aortic valve insertion, percutaneous ventricular assist device insertion). When used to maintain a life-saving device for a duration exceeding procedural support, a specialty sheath covers the graft to maintain sterility. Once access to the vessel via the graft is no longer needed, the graft can be ligated and transected with the graft closure device and graft cutting device, respectively. One patient population for the system are those with heart failure or those undergoing heart procedures. There are approximately 100,000 cases annually in the US that could benefit from the use of the system. Currently, these procedures require a surgeon to perform and have a morbidity approaching 25-30% due to bleeding, nerve damage, and repeat surgery. In some embodiments, the device reduces the skill required for an end-to-side anastomosis with graft making, compared to typical devices. It is also amenable to use by non-surgeons (e.g., interventional cardiologists) and mitigates complications that patients experience with open surgical exposure of vessels for graft anastomosis, compared to typical devices. It can also reduce the time required for the procedure, the resources required (e.g. less support staff in a catheterization lab vs. the operating room) and expands the opportunity for care to hospitals where surgeons are not readily available, compared to typical devices.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A percutaneous access graft system comprising:
a vascular graft having a flared end configured for end-to-side anastomosis placement on a vessel;
a sheath engaging the vascular graft to enable prolonged use and maintenance of the graft without exposing the vascular graft to the environment; and
a closure device configured to percutaneously ligate the vascular graft.

2. The percutaneous access graft system of claim 1, wherein the sheath further comprises an outer sheath and an inner sheath, the inner sheath being removably coupled to the outer sheath.

3. The percutaneous access graft system of claim 2, wherein at least a proximal portion of said vascular graft is positioned between said outer sheath and said inner sheath.

4. The percutaneous access graft system of claim 3, further comprising a locking cap affixing the outer sheath to the inner sheath to form sterile chamber holding at least the proximal portion of the vascular graft.

5. The percutaneous access graft system of claim 3, wherein the inner sheath is configured to facilitate insertion and removal of medical devices through vascular graft.
